(19) United States
(12) Patent Application Publication
(10) Pub. No.: US 2021/0024531 A1
(43) Pub. Date: Jan. 28, 2021

(54) PROCESS FOR PRODUCING HERBICIDAL PYRIDAZINONE COMPOUNDS

(71) Applicant: Syngenta Participations AG, Basel (CH)

(72) Inventors: HELMARS SMITS, Münchwilen (CH); RAPHAEL DUMEUNIER, Stein (CH); EDOUARD GODINEAU, Stein (CH); MATTHIAS LEHMANN, Stein (CH); HARRY JOHN MILNER, Münchwilen (CH); ALAN JAMES ROBINSON, Münchwilen (CH)

(73) Assignee: Syngenta Participations AG, Basel (CH)

(21) Appl. No.: 16/757,255

(22) PCT Filed: Oct. 16, 2018
(86) PCT No.: PCT/EP2018/078303

§ 371 Date: Apr. 17, 2020

(30) Foreign Application Priority Data
Oct. 18, 2017 (GB) 1717078.8

(51) Int. Cl. C07D 491/04; C07D 237/14; C07C 251/72; A01N 33/26; A01N 43/90

(52) U.S. Cl. CPC C07D 491/04; C07D 237/14; A01N 43/90; A01N 33/26; C07C 251/72

(57) ABSTRACT

The present invention provides, inter alia, a process for producing a compound of Formula (I): wherein A¹, R¹, R², R³, R⁴, R⁵ and R⁶ are as defined herein. The present invention further provides intermediate compounds utilised in said process, and methods for producing said intermediate compounds.

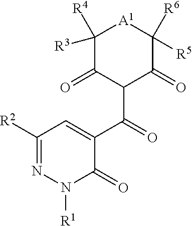

The present invention relates to a process for producing herbicidal pyridazinone compounds. Such compounds are known, for example, from WO 2012/136703 and WO2017/178582. As explained therein, such compounds are typically prepared by reacting an acid chloride of the corresponding pyridazinone with cyclohexanedione in the presence of a base to first make an enol ester which is then rearranged to the pyridazinone triketone using a catalytic amount of cyanide source, typically acetone cyanohydrin. This reaction is understood to proceed via an intermediate acyl cyanide as described in, for example, Montes, I. F.; Burger, U. Tetr. Lett. 1996, 37, 1007. However the yields achieved using such a cyanide rearrangement procedure are not ideal for a large scale production and the use of toxic cyanides in commercial manufacturing remains undesirable. Therefore a new, more efficient synthesis method not involving the use of cyanide ions is desired.

Surprisingly, it has now been found that the herbicidal pyridazinones can actually be produced in the absence of a cyanide catalysed rearrangement step. Further optimisation of the process allows an efficient, cyanide-free synthesis of such compounds.

Thus, according to the present invention there is provided a process for producing a compound of Formula (I):

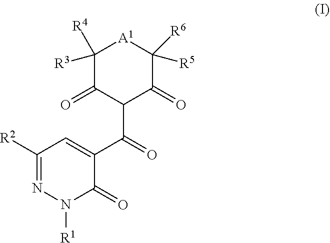

wherein
R¹ is selected from the group consisting of hydrogen, C₁-C₆alkyl, C₁-C₆haloalkyl, C₁-C₃alkoxyC₁-C₃alkyl-, C₁-C₃alkoxyC₂-C₃alkoxyC₁-C₃alkyl-, aryl and a 5 or 6-membered heteroaryl, wherein the heteroaryl contains one to three heteroatoms each independently selected from the group consisting of oxygen, nitrogen and sulphur, and wherein the aryl and heteroaryl component may be optionally substituted;
R² is C₁-C₆ alkyl or C₃-C₆ cycloalkyl;
A¹ is selected from the group consisting of O, C(O) and (CR⁷R⁸); and
R⁴, R⁶, R⁷ and R⁸ are each independently selected from the group consisting of hydrogen and C₁-C₄alkyl;
R³ and R⁵ are each independently selected from the group consisting of hydrogen and C₁-C₄alkyl or together may form a C₁-C₃alkylene (e.g ethylene) chain;
the process comprising
   (i) reacting a compound of Formula (XII)

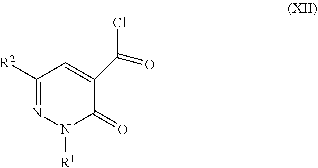

wherein R¹ and R² are as defined with regard to Formula (I) above, with a compound of Formula (XIII)

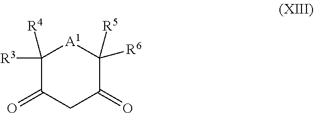

wherein A¹ and R³, R⁴, R⁵ and R⁶ are as defined with regard to Formula (I) above;
to give a compound of Formula (XIV)

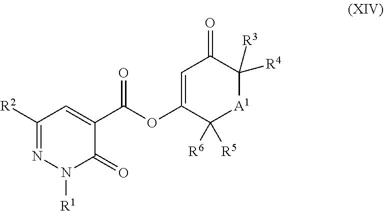

(ii) converting the compound of Formula (XIV) to a compound of Formula (XV)

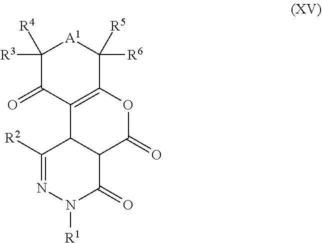

and
(iii) converting the compound of Formula (XV) to a compound of Formula (I)
   wherein the process is carried out in the presence of a base and in the absence of cyanide ions.

C₁-C₆alkyl and C₁-C₄alkyl groups referred to above include, for example, methyl (Me, CH₃), ethyl (Et, C₂H₅), n-propyl (n-Pr), isopropyl (i-Pr), n-butyl (n-Bu), isobutyl (i-Bu), sec-butyl and tent-butyl (t-Bu).

Halogen (or halo) includes fluorine, chlorine, bromine and iodine.

C₁-C₆haloalkyl includes, for example, fluoromethyl-, difluoromethyl-, trifluoromethyl-, chloromethyl-, dichloromethyl-, trichloromethyl-, 2,2,2-trifluoroethyl-, 2-fluoroethyl-, 2-chloroethyl-, pentafluoroethyl-, 1,1-difluoro-2,2,2-trichloroethyl-, 2,2,3,3-tetrafluoroethyl-, 2,2,2-trichloroethyl-, heptafluoro-n-propyl and perfluoro-n-hexyl. C₁-C₄haloalkyl includes, for example, fluoromethyl-, difluoromethyl-, trifluoromethyl-, chloromethyl-, dichloromethyl-, trichloromethyl-, 2,2,2-trifluoroethyl-, 2-fluoroethyl-, 2-chloroethyl-, pentafluoroethyl-, 1,1-difluoro-2,2,2-trichloroethyl-, 2,2,3,3-tetrafluoroethyl-, 2,2,2-trichloroethyl- and heptafluoro-n-propyl-. Preferred C₁-C₆haloalkyl groups are fluoroalkyl groups, especially diflluoroalkyl and trifluoroalkyl groups, for example, difluoromethyl and trifluoromethyl.

C₃-C₆ cycloalkyl group includes, for example, cyclopropyl, cyclobutyl, cyclopentyl or cyclohexyl.

C₁-C₃alkoxyC₁-C₃alkyl- includes, for example, methoxymethyl, methoxyethyl, ethoxymethyl, ethoxyethyl, n-propoxymethyl, n-propoxyethyl, isopropoxymethyl or isopropoxyethyl.

C₁-C₃alkoxyC₂-C₃alkoxyC₁-C₃alkyl- includes, for example, methoxyethoxymethyl-. Nitro, as used herein, refers to the group —NO₂.

Aryl, as used herein, refers to an unsaturated aromatic carbocyclic group of from 6 to 10 carbon atoms having a single ring (e. g., phenyl) or multiple condensed (fused) rings, at least one of which is aromatic (e.g., indanyl, naphthyl). Preferred aryl groups include phenyl, naphthyl and the like. Most preferably, the aryl group is a phenyl group. The phenyl may be unsubstituted or in mono- or poly-substituted form, in which case the substituents may, as desired, be in the ortho-, meta- and/or para-position(s).

A 5- or 6-membered heteroaryl group, wherein the heteroaryl contains one to three heteroatoms each independently selected from the group consisting of oxygen, nitrogen and sulphur includes, for example, furanyl, thiophenyl, thiazolyl, oxazolyl, isoxazolyl, thiazolyl, pyrazolyl, isothiazolyl, pyridyl, pyridazinyl, pyrazinyl, pyrimidinyl and triazolyl. The heteroaryl component may be optionally mono or poly substituted as described.

Where the aryl or heteroaryl components described above are substituted, the one or more substituents are preferably selected from the group consisting of halo, C₁-C₄alkyl, C₁-C₄haloalkyl, C₁-C₃ alkoxy, cyano and nitro.

In one embodiment of the present invention, R¹ is an optionally substituted heteroaryl.

In another embodiment of the present invention, R¹ is an optionally substituted phenyl.

More preferably, R¹ is phenyl optionally substituted by one or two substituents independently selected from the group consisting of halo, C₁-C₄alkyl, C₁-C₄haloalkyl, C₁-C₃ alkoxy, cyano and nitro. Most preferably R¹ is 3,4-dimethoxyphenyl-.

In one embodiment of the present invention, R² is methyl.

In a particularly preferred embodiment of the present invention, R¹ is 3,4-dimethoxyphenyl and R² is methyl.

In one embodiment of the present invention A¹ is CR⁷R⁸ and R³, R⁴, R⁵, R⁶, R⁷ and R⁸ are hydrogen. Thus, in a particularly preferred embodiment of the present invention the compound of Formula (XIII) is cyclohexanedione.

In one embodiment of the present invention, A¹ is CR⁷R⁸ and R⁴, R⁶, R⁷ and R⁸ are hydrogen and R³ and R⁵ together form an ethylene chain.

In a particularly preferred embodiment of the present invention, A¹ is CR⁷R⁸ and R³, R⁴, R⁵, R⁶, R⁷ and R⁸ are hydrogen, R¹ is 3,4-dimethoxyphenyl and R² is methyl.

The process of the present invention can be carried out in separate process steps, wherein the intermediate compounds can be isolated at each stage. Alternatively, the process can be carried out in a one-step procedure wherein the intermediate compounds produced are not isolated. Thus, it is possible for the process of the present invention to be conducted in a batch wise or continuous fashion.

In a preferred embodiment of the present invention the process is carried out using an excess of a compound of Formula (XIII) and/or base. Typically, this can be achieved by taking a mixture of the compound of Formula (XIII) and the relevant base and then adding the compound of Formula (XII) to said mixture in order to produce the compound of Formula (I).

The present invention further provides a process as referred to above, wherein the compound of Formula (XII) is produced by
(i) reacting together a compound of Formula (V)

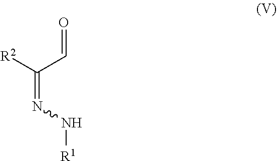

wherein R¹ is aryl or a 5 or 6-membered heteroaryl and R² are as defined with regard to Formula (I) above; with a compound of formula (XVI)

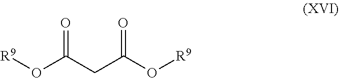

wherein each R⁹ is independently a C₁-C₆alkyl, preferably methyl or ethyl to give a compound of formula (VI)

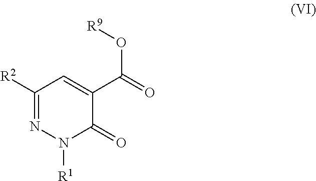

(ii) hydrolysing the compound of Formula VI to a compound of Formula (IX)

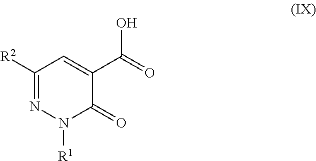

and
(iii) converting the compound of Formula (IX) to the corresponding acid chloride of Formula (XII)

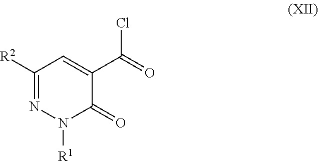

The present invention still further provides a process wherein the compound of Formula (XII) is produced by
(i) reacting together a compound of Formula (III)

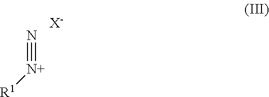

wherein R¹ is aryl or a 5 or 6-membered heteroaryl as defined with regard to Formula (I) above and X is selected from the group consisting of Cl, Br and HSO₄ with a compound of Formula (X)

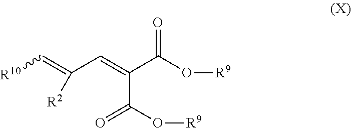

wherein R² is as defined above, R¹⁰ is NMe₂, NEt₂, OH or C₁-C₃alkoxy and each R⁹ is independently a C₁-C₆alkyl, preferably methyl or ethyl to give a compound of Formula (XI)

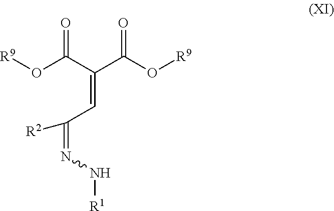

(ii) cyclizing the compound of Formula (XI) to a compound of Formula (VI)

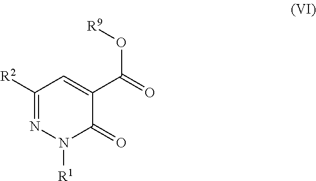

(iii) hydrolysing the compound of Formula (VI) to a compound of Formula (IX)

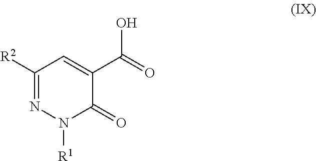

and
(iv) converting the compound of Formula (IX) to the corresponding acid chloride of Formula (XII)

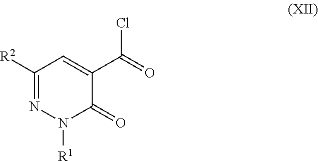

In a preferred embodiment of the processes described above R¹ is 3,4-dimethoxyphenyl and R² is methyl.

The present invention still further provides intermediate compounds afforded by the processes of the present invention. Thus, according to the present invention there is provided a compound of Formula (XV)

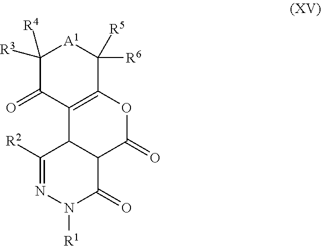

wherein R¹, R², R³, R⁴, R⁵, R⁶ and A¹ are as defined for a compound of Formula (I).

The present invention still further provides a compound of Formula (XVa) including all stereoisomers thereof.

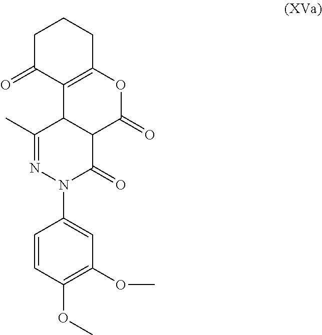 [SMILES: COc1ccc(N2N=C(C)C3C4=C(CCCC4=O)OC(=O)C3C2=O)cc1OC]

The present invention further provides a compound of Formula (Va)

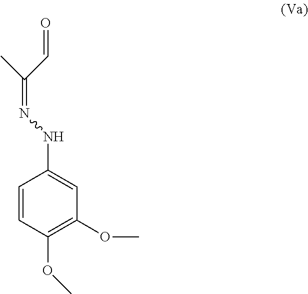 [SMILES: COc1ccc(NN=C(C)C=O)cc1OC]

The present invention still further provides a compound of Formula (XIa)

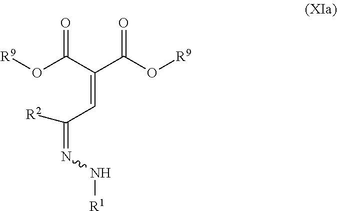

wherein R⁹ are both methyl or ethyl, R² is methyl and R¹ is 3,4-dimethoxyphenyl-.

Scheme 1 below outlines the subject matter of the invention in more detail. The substituent definitions are the same as defined above.

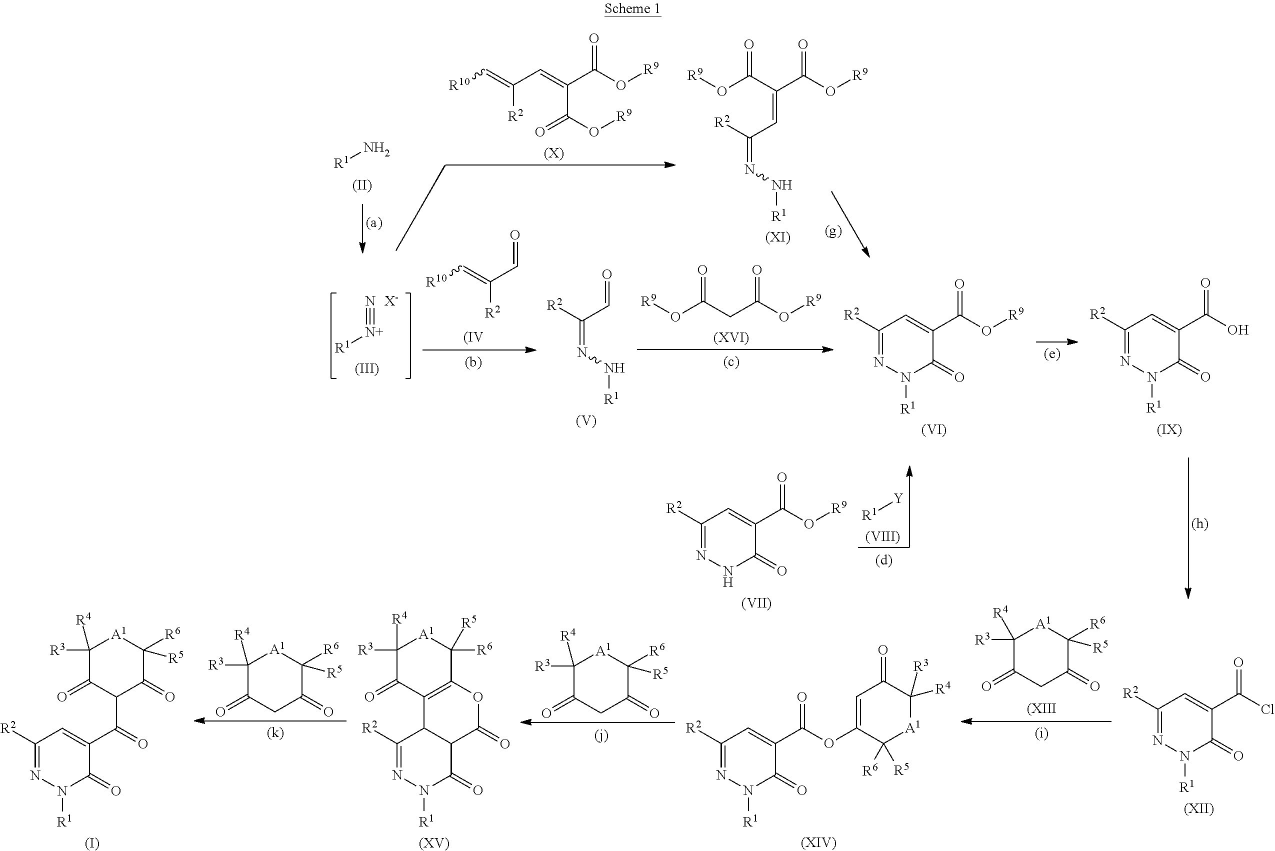

### Step (a)

Compound of Formula (III) is typically prepared by diazotation of a compound of formula (II) wherein R¹ is aryl or a 5 or 6-membered heteroaryl as defined with regard to Formula (I) above, wherein the heteroaryl contains one to three heteroatoms each independently selected from the group consisting of oxygen, nitrogen and sulphur, and wherein the aryl and heteroaryl component may be optionally substituted using a suitable diazotating agent in the presence of an acid. Typically, this is achieved using NaNO₂ in water and in the presence of a strong mineral acid such as HCl, HBr, HBF₄ and H₂SO₄. The most preferred acid is H₂SO₄. Typically a compound of Formula (III) is not isolated but kept in the solution and engaged directly into the next step.

### Step (b)

The compound of Formula (V) is typically prepared by reacting a compound of Formula (III) with a compound of Formula (IV) wherein R² is as defined above for a compound of formula (I) and R¹⁰ is NMe₂, NEt₂, OH, C₁-C₃alkoxy as for example described in Shvedov, V. I.; Galstukhova, N. B.; Pankina, Z. A.; Zykova, T. N.; Lapaeva, N. B.; Pershin, G. N. Khim.Farm.Zh. 1978, 12, 88 in the presence of a base. Suitable bases include, but are not limited to water soluble inorganic bases such as NaOH, KOH, Na₂CO₃, K₂CO₃, NaH₂PO₄, Na₂HPO₄, Na₃PO₄, NaHCO₃ and NaOAc as well as tertiary amine bases such as Et₃N and iPr₂NEt. The most preferred bases are NaOAc and Na₂HPO₄.

The reaction between compounds of Formulae (III) and (IV) is preferably carried out in the presence of a solvent. The most suitable solvent is water.

The reaction can be carried out at a temperature from −20° C. to 50° C., preferably from 0° C. to 25° C.

### Step (c)

The compound of Formula (VI) is typically prepared by reacting a compound of Formula (V) with a compound of Formula (XVI) e.g diethylmalonate in the presence of a secondary amine catalyst as for example described in Jolivet, S.; Texier-Boullet, F.; Hamelin, J.; Jacquault, P. Heteroatom Chem. 1995, 6, 469. Suitable catalysts include, but are not limited to piperidine, morpholine, Et₂NH and iPr₂NH. The most preferred catalyst is piperidine. The amount of secondary amine catalyst is between 0.05 and 1 equivalent, more preferably between 0.2 and 0.5 equivalents.

Optionally the reaction is run in the presence of an acid as a catalyst. Suitable acids include, but are not limited to AcOH and TFA. The most preferred acid catalyst is AcOH. The amount of the acid is from 0.05 to 1 equivalent, more preferably from 0.2 to 0.5 equivalents.

The reactions between compounds of Formula (V) and e.g diethylmalonate are preferably carried out in the presence of a solvent. Suitable solvents include, but are not limited to ethanol, methanol, toluene and xylenes. The most preferred solvents are toluene and ethanol. Optionally the reaction can also be carried out using the compound of Formula (XVI) e.g diethylmalonate as a solvent.

The reaction can be carried out at a temperature from 20° C. to 120° C., preferably from 50° C. to 90° C.

### Step (d)

Alternatively the compound of Formula (VI) can be prepared by reacting a compound of formula (VII) with a compound of Formula (VIII) wherein R¹ is C₁-C₆alkyl, C₁-C₆haloalkyl, C₁-C₃alkoxyC₁-C₃alkyl-, C₁-C₃alkoxyC₂-C₃alkoxyC₁-C₃alkyl- and Y=Br, Cl or I in the presence of a base using methods known to a person skilled in the art.

### Step (e)

The compound of Formula (IX) is typically prepared by hydrolysing compound of Formula (VI) using methods known to a person skilled in the art. The hydrolysis is typically performed using an aqueous base, for example aqueous NaOH or KOH.

### Step (f)

Alternatively, compounds of Formula (IX) can be prepared by first reacting a compound of Formula (III)

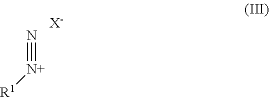

wherein R¹ is aryl or a 5 or 6-membered heteroaryl as defined with regard to Formula (I) above and X is selected from the group consisting of Cl, Br and HSO₄; with a compound of Formula (X)

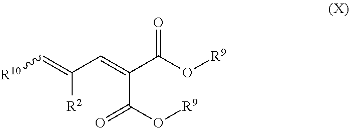

wherein R² is as defined above for a compound of Formula (I), R¹⁰ is NMe₂, NEt₂, OH or C₁-C₃alkoxy and R⁹ is C₁-C₆alkyl and which can be prepared as described in WO2002/034710 in the presence of a base to produce a compound of Formula (XI)

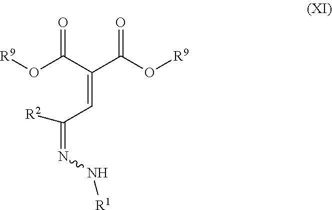

wherein R¹ is as defined above for a compound of Formula (III), and R² and R⁹ is as defined above for a compound of Formula (X). Suitable bases include, but are not limited to water soluble inorganic bases such as NaOH, KOH, Na₂CO₃, K₂CO₃, NaH₂PO₄, Na₂HPO₄, Na₃PO₄, NaHCO₃, NaOAc as well as tertiary amine bases such as Et₃N and iPr₂NEt. The most preferred bases are NaOAc and Et₃N.

The reaction between compounds of Formula (III) and Formula (X) are preferably carried out in the presence of a solvent. The most suitable solvent is water.

The reaction can be carried out at a temperature from −20° C. to 50° C., preferably from 0° C. to 25° C.

### Step (g)

The compound of Formula (VI) is typically prepared by heating a compound of Formula (XI) in a suitable solvent. Suitable solvents include, but are not limited to toluene, xylenes, THF, dioxane and 1,2-dichloroethane. The most preferred solvent is toluene.

Alternatively the compound of Formula (VI) is prepared by reaction a compound of Formula (XI) with a suitable base. Suitable bases include, but are not limited to alkali metal hydroxides and carbonates such as NaOH, KOH and Na₂CO₃ as well as tertiary amine bases such as Et₃N, DMAP and iPr₂NEt.

When no base is used the reaction can be carried out at a temperature from 40° C. to 120° C., preferably from 70° C. to 100° C. When base is used the reaction can be carried out at a temperature from −10° C. to 50° C., most preferably at ambient temperature.

### Step (h)

The compound of Formula (IX) can be converted to acid chloride of Formula (XII) using chlorinating procedures well known to the skilled person. Typical chlorinating agents include, for example, thionyl chloride, oxalyl chloride, phosphorous oxychloride, diphosgene, triphosgene and phosgene.

### Step (i)

The compound of Formula (XIV) is typically prepared by reacting a compound of formula (XII) with a compound of Formula (XIII) in the presence of a base. Suitable bases include, but are not limited to organic amine bases such as N,N-dimethyl aniline, triethylamine, di-isopropylethyl amine, pyridine, DBU and 2,6-lutidine as well as inorganic bases such as K₂CO₃, NaOH, KOH and NaHCO₃. The most preferable bases are N,N-dimethyl aniline and triethylamine. The amount of a base is typically between 1.0 and 2.5 equivalents, preferably between 1.0 and 1.5 equivalents.

The reaction between compounds of Formula (XII) and (XIII) are preferably carried out in the presence of a solvent. Suitable solvents include, but are not limited to polar aprotic solvents such as acetonitrile, dioxane, 1,2-dichloroethane, dichloromethane and chloroform. The most preferred solvents are acetonitrile and 1,2-dichloroethane.

The reaction can be carried out at a temperature from −40° C. to 70° C. When bases insufficiently strong to deprotonate a compound of Formula (XIII) are used the preferred temperature is from 0° C. to 25° C. When bases which fully deprotonate compounds of Formula (XIII) are used the preferred temperature is from -20° C. to 0° C.

### Step (j)

The compound of Formula (XV) is typically prepared by reacting a compound of Formula (XIV) with a catalytic amount of a base and optionally a catalytic amount of a compound of Formula (XIII). Suitable bases include, but are not limited to amine bases sufficiently strong to deprotonate a compound of Formula (XIII) such as triethylamine, 2,6-lutidine, pyridine, diisopropylethyl amine, DMAP and DBU as well as inorganic bases such as K₂CO₃, NaOH, KOH and Na₂CO₃. The amount of a base is from 0.05 to 1.5 equivalents, preferably from 0.2 to 1.2 equivalents.

When a catalytic amount of a compound of Formula (XIII) is used the amount is typically from 0.02 to 0.8 equivalents, preferably from 0.1 to 0.3 equivalents.

The reaction of a compound of Formula (XIV) with a base and optionally with a compound of Formula (XIII) are preferably carried out in the presence of a solvent. Suitable solvents include, but are not limited to polar aprotic solvents such as acetonitrile, dioxane, 1,2-dichloroethane, dichloromethane and chloroform. The most preferred solvents are acetonitrile and 1,2-dichloroethane.

The reaction can be carried out at a temperature from −10° C. to 70° C., more preferable from −5° C. to 25° C.

### Step (k)

The compound of Formula (I) is typically prepared by reacting a compound of Formula (XV) with a catalytic amount of a compound of Formula (XIII) in the presence of a base.

The amount of a compound of Formula (XIII) is from 0.02 to 0.8 equivalents, preferably from 0.1 to 0.3 equivalents. Suitable bases include, but are not limited to amine bases sufficiently strong to deprotonate a compound of Formula (XIII) such as triethylamine, 2,6-lutidine, pyridine, diisopropylethyl amine, DMAP and DBU as well as inorganic bases such as NaOH, KOH, Na₂CO₃ and K₂CO₃. The most preferred base is triethylamine.

The reaction between compounds of Formula (XV) and (XIII) are preferably carried out in the presence of a solvent. Suitable solvents include, but are not limited to polar aprotic solvents such as acetonitrile, dioxane, 1,2-dichloroethane, dichloromethane and chloroform. The most preferred solvents are acetonitrile and 1,2-dichloroethane.

The reaction can be carried out at a temperature from 0° C. to 100° C., more preferable between 20° C. and 70° C.

Optionally the steps (i), (j) and (k) can be carried out in a single step without isolating any of the intermediates.

### EXAMPLES

The following non-limiting Examples outline the subject matter of the invention in more detail. The substituent definitions are the same as defined above.

The following abbreviations were used in this section: s=singlet; br s=broad singlet; d =doublet; dd=double doublet; dt=double triplet; t=triplet, tt=triple triplet, q=quartet, quin =quintuplet, sept=septet; m=multiplet; RT=retention time, MH⁺=molecular mass of the molecular cation.

¹H NMR spectra were recorded at 400 MHz and chemical shifts are given in ppm.

### Example 1

### (2Z)-2-[(3,4-Dimethoxyphenyl)hydrazono]propanal

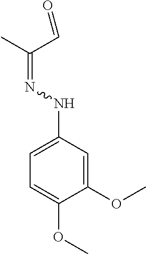 [SMILES: COc1ccc(NN=C(C)C=O)cc1OC]

A 5 l double jacketed reactor was charged with water (1.2 l) and cooled to 5° C. Concentrated sulfuric acid (104 ml, 1.90 mol) was added slowly while keeping the temperature below 25° C. When the internal temperature has again reached 5° C. 3,4-dimethoxyaniline (198.0 g, 1.27 mol) was added portion wise. A solution of sodium nitrite (88.3 g, 1.27 mol) in water (0.25 l) was added to the dark violet suspension over 40 min while keeping the internal temperature below 5° C. The reaction mixture was stirred at 0° C. for 90 min followed by addition of the solution of 3-dimethylamino-2-methyl-2-propanal (137.2 g, 1.15 mol) and NaOAc (105.0 g, 1.27 mol) in water (0.75 l) over 1 h while keeping the internal temperature below 5° C. After the addition was finished the temperature of the reactor jacket was raised to 0° C. and afterwards every 30 min again 5° C. After 2.5 h (internal temperature 20° C.) full conversion has been achieved. The now black suspension was transferred into 5 l Erlenmeyer flask and the reactor was washed with water (2 l) to remove most of the remaining precipitate. The solid product was filtered off, washed on filter with water (1.5 l) and dried to constant weight at 50° C. and high vacuum for 40 h to yield (2Z)-2-[(3,4-dimethoxyphenyl)hydrazono]propanal (199 g, 92% purity, 71% yield) as a brick red solid. This material was sufficiently pure to be used in the next step. Upon standing for 16 h another portion of the product precipitated out of the aqueous phase and was also filtered, washed and dried in vacuum to provide the second batch of (2Z)-2-[(3,4-dimethoxyphenyl)hydrazono]propanal (43.7 g, 70% purity, 12% yield; 83% yield for combined both batches).

¹H NMR (400 MHz, CDCl₃): δ 9.49 (s, 1H), 8.10 (br s, 1H), 7.00 (d, J=2.6 Hz, 1H), 6.86 (d, J=8.4 Hz, 1H), 6.70 (dd, J=8.6, 2.4 Hz, 1H), 3.94 (s, 3H), 3.88 (s, 3H), 1.98 (s, 3H).

### Example 2

### Ethyl 2-(3,4-dimethoxyphenyl)-6-methyl-3-oxo-pyridazine-4-carboxylate

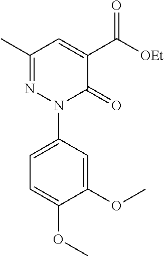 [SMILES: CCOC(=O)c1cc(C)nn(-c2ccc(OC)c(OC)c2)c1=O]

To a solution of piperidine (0.062 ml, 0.628 mmol) in toluene (3.0 ml) was added acetic acid (0.036 ml, 0.628 mmol). After stirring for 10 min (2Z)-2-[(3,4-dimethoxyphenyl) hydrazono]propanal (0.300 g, 93% purity, 1.26 mmol) was added followed by diethyl malonate (0.23 ml, 1.51 mmol). The resulting dark red reaction mixture was heated at 90° C. for 3 h, cooled to rt and evaporated to dryness under reduced pressure to afford ethyl 2-(3,4-dimethoxyphenyl)-6-methyl-3-oxo-pyridazine-4-carboxylate (0.564 g) as a red oil. Quantitative NMR analysis using trimethoxybenzene as an internal standard indicates purity of 67% (94% yield).

¹H NMR (400 MHz, CDCl₃): δ 7.67 (s, 1H), 7.15-7.10 (m, 2H), 6.96-6.92 (m, 1H), 4.42 (q, J=7.3 Hz, 2H), 3.92 (s, 3H), 3.90 (s, 3H), 2.44 (s, 3H), 1.40 (t, J=7.2 Hz, 3H).

### Example 3

### 2-(3,4-Dimethoxyphenyl)-6-methyl-3-oxo-pyridazine-4-carboxylic acid

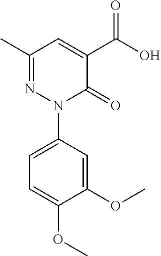 [SMILES: COc1ccc(-n2nc(C)cc(C(=O)O)c2=O)cc1OC]

A 5 l double jacketed reactor was charged with toluene (1.25 l). Piperidine (31.0 ml, 0.31 mol) was added followed by acetic acid (18.0 ml, 0.31 mol) resulting in a formation of white precipitate. (2Z)-2-[(3,4-dimethoxyphenyl)hydrazono]propanal (150 g, 92% purity, 0.621 mol) was added affording a dark red suspension. Diethyl malonate (114 ml, 0.745 mol) was added and the reaction mixture was heated to 96° C. (reflux). After stirring for 8 h the reaction mixture was cooled to 20° C. and stirred for further 16 h. 2M NaOH (0.621 l, 1.24 mol) was slowly added while keeping the internal temperature below 30° C. After stirring for 2 h extra water (0.5 l) was added. After stirring for further 1 h water (1.75 l) and toluene (0.20 l) was added to dissolve all precipitates. Stirring was then stopped and layers separated. Organic layer was washed with water (2×0.7 l) and combined aqueous layers were washed with EtOAc (2×0.4 l). The aqueous layer was then slowly poured into 2M HCl (1.00 l, 2.00 mol). A yellow solid precipitates from the mixture immediately. After the addition was finished (15 min) the mixture was stirred for additional 20min. The precipitate was filtered off, washed on filter with water and dried at high vacuum and 65° C. till constant weight affording ethyl 2-(3,4-dimethoxyphenyl)-6-methyl-3-oxo-pyridazine-4-carboxylate (153.8 g) as a yellow powder. Quantitative NMR analysis using trimethoxybenzene as an internal standard indicates purity of 95% (81% isolated yield).

¹H NMR (400 MHz, CDCl₃): δ 13.98 (br s, 1H), 8.18 (s, 1H), 7.16 (dd, J=8.6, 2.5 Hz, 1H), 7.09 (d, J=2.4 Hz, 1H), 6.99 (d=8.6 Hz, 1H), 3.95 (s, 3H), 3.93 (s, 3H), 2.55 (s, 3H).

### Example 4

### Dimethyl 2-[(2Z)-2-[(3,4-dimethoxyphenyl)hydrazono]propylidene] propanedioate

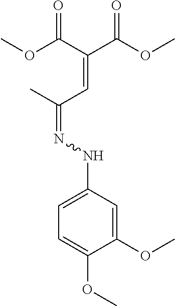

To a suspension of 3,4-dimethoxyaniline (63 mg, 0.42 mmol) in H₂O (1.51 ml) was added sulfuric acid (35 μl, 0.62 mmol) at 23° C. NaNO₂ (aq. sol, 1.0 M, 0.42 ml, 0.42 mmol) was added within 30 s. To this mixture a solution of dimethyl 2-[(E)-3-(dimethylamino)-2-methyl-prop-2-enylidene]propanedioate (100 mg, 86% purity, 0.38 mmol) and NaOAc (35 mg, 0.42 mmol) in H₂O/MeOH (6 ml, 1:1, v:v) was added in one portion. The mixture was stirred for 5 min at 23° C. H₂O (20 ml) was added to the reaction mixture and it was extracted with EtOAc (3×20 ml). The combined organic phases were dried over Na₂SO₄ and the solvent was evaporated to afford dimethyl 2-[(2Z)-2-[(3,4-dimethoxyphenyl)hydrazono]-propylidene]-propanedioate (166 mg) as a red solid. Quantitative NMR analysis using mesitylene as an internal standard indicates purity of 63% (82% yield).

¹H NMR (400 MHz, CDCl₃): δ=2.01 (s, 3H), 3.82 (s, 3H), 3.85 (s, 3H), 3.88 (s, 3H), 4.01 (s, 3H), 6.52-6.55 (m, 1H), 6.98-6.99 (m, 1H), 7.27 (s, 1H), 7.37 (s, 1H), 7.73 (bs, 1H).

### Example 5

### Methyl 2-(3,4-dimethoxyphenyl)-6-methyl-3-oxo-pyridazine-4-carboxylate

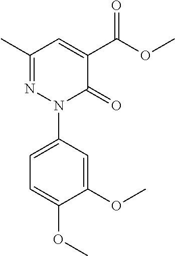

A solution of dimethyl 2-[(2Z)-2-[(3,4-dimethoxyphenyl)hydrazono]propylidene] propanedioate (121 mg, 63% purity, 0.23 mmol) in toluene (0.91 ml) was heated to 90° C. for 3.5 h. The solvent was evaporated to afford methyl 2-(3,4-dimethoxyphenyl)-6-methyl-3-oxo-pyridazine-4-carboxylate (85.8 mg) as a white solid. Quantitative NMR analysis using mesitylene as an internal standard indicates purity of 63% (78% yield).

¹H-NMR (400 MHz, CDCl₃): δ=2.46 (s, 3H), 3.92 (s, 3H), 3.94 (s, 3H), 3.97 (s, 3H), 6.94-6.96 (m, 1H), 7.13-7.16 (m, 2H), 7.72 (s, 1H).

### Example 6

### 2-(3,4-Dimethoxyphenyl)-6-methyl-3-oxo-pyridazine-4-carbonyl chloride

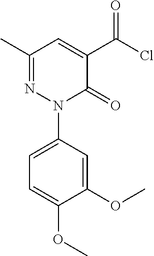 [SMILES: COc1ccc(-n2nc(C)cc(C(=O)Cl)c2=O)cc1OC]

To a suspension of 2-(3,4-dimethoxyphenyl)-6-methyl-3-oxo-pyridazine-4-carboxylic acid (10.0 g, 97% purity, 33.4 mmol) in dichloromethane (50 ml) was added DMF (0.052 ml, 0.67 mmol). Oxalyl chloride (3.87 ml, 43.4 mmol) was then added slowly (strong gas evolution). After stirring for 2 h at ambient temperature the reaction mixture was evaporated to dryness under reduced pressure to afford 2-(3,4-dimethoxyphenyl)-6-methyl-3-oxo-pyridazine-4-carbonyl chloride (10.77 g) as a dark brown solid. Quantitative NMR analysis using trimethoxybenzene as an internal standard indicates purity of 95% (99% yield).

¹H NMR (400 MHz, CDCl₃): δ 7.88 (s, 1H), 7.16-7.11 (m, 2H), 6.94 (d, J=8.1 Hz, 1H), 3.93 (s, 3H), 3.90 (s, 3H), 2.51 (s, 3H).

### Example 7

### (3-Oxocyclohexen-1-yl) 2-(3,4-dimethoxyphenyl)-6-methyl-3-oxo-pyridazine-4-carboxylate

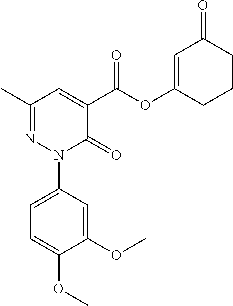 [SMILES: COc1ccc(-n2nc(C)cc(C(=O)OC3=CC(=O)CCC3)c2=O)cc1OC]

To a solution of 1,3-cyclohexadione (0.647 g, 5.77 mmol) in 1,2-dichloroethane (40 ml) was added triethylamine (0.93 ml, 6.63 mmol) at −15° C. resulting in a clear colourless solution. A solution of 2-(3,4-dimethoxyphenyl)-6-methyl-3-oxo-pyridazine-4-carbonyl chloride (1.963 g, 85% purity, 5.42 mmol) in 1,2-dichloroethane (20 ml) was added dropwise while keeping the internal temperature below −12° C. After stirring for 2 h the reaction was quenched by addition of 1M HCl (40 ml) and allowed to warm to ambient temperature. Layers were separated, organic phase washed with 1M HCl (40 ml), water (40 ml) and brine (40 ml) and dried over anhydrous Na₂SO₄. Concentration under reduced pressure afforded (3-oxocyclohexen-1-yl) 2-(3,4-dimethoxyphenyl)-6-methyl-3-oxo-pyridazine-4-carboxylate (2.50 g) as an amber coloured oil. Quantitative NMR analysis using trimethoxybenzene as an internal standard indicates purity of 83% (99% yield).

¹H NMR (400 MHz, CDCl₃): δ 7.79 (s, 1H), 7.13 (dd, J=8.4, 2.4 Hz, 1H), 7.10 (d, J=2.5 Hz, 1H), 6.95 (d, J=8.7 Hz, 1H), 6.04 (t, J=1.3 Hz, 1H), 3.93 (s, 3H), 3.91 (s, 3H), 2.69 (td, J=6.1, 1.3 Hz, 2H), 2.49 (s, 3H), 2.48-2.43 (m, 2H), 2.12 (quin, J=6.5 Hz, 2H).

Alternatively (3-oxocyclohexen-l-yl) 2-(3,4-dimethoxyphenyl)-6-methyl-3-oxo-pyridazine-4-carboxylate could be prepared by the following procedure:

To a solution of 2-(3,4-dimethoxyphenyl)-6-methyl-3-oxo-pyridazine-4-carbonyl chloride (3.28 g, 94% purity, 9.99 mmol) in 1,2-dichloroethane (35 ml) was added 1,3-cyclohexadione (1.50 g, 97% purity, 13.0 mmol) followed by N,N-dimethylaniline (3.2 ml, 25.0 mmol). The deep red solution was stirred at ambient temperature for 1 h. The reaction mixture was then diluted with 1,2-dichloroethane (40 ml) and washed with 1M HCl, sat. aq. NaHCO₃ and brine. The organic layer was dried over anhydrous Na₂SO₄ and evaporated under reduced pressure to produce a black gummy residue. Diethyl ether (10 ml) was added and the resulting suspension was stirred vigorously for 4 h. The resulting precipitate was filtered off and washed on filter with a minimum amount of diethyl ether. Drying of precipitate in high vacuum afforded (3-oxocyclohexen-1-yl) 2-(3,4-dimethoxyphenyl)-6-methyl-3-oxo-pyridazine-4-carboxylate (2.81 g, 97% purity, 71% isolated yield) as a beige solid.

### Example 8

### 3-(3,4-Dimethoxyphenyl)-1-methyl-7,8,9,10b-tetrahydro-4aH-chromeno[3,4-d]pyridazine-4,5,10-trione

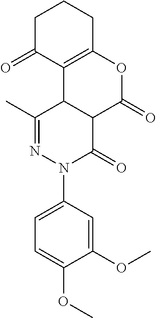 [SMILES: COc1ccc(N2N=C(C)C3C4=C(CCCC4=O)OC(=O)C3C2=O)cc1OC]

To a solution of (3-oxocyclohexen-1-yl) 2-(3,4-dimethoxyphenyl)-6-methyl-3-oxo-pyridazine-4-carboxylate (0.444 g, 96% purity, 1.00 mmol) and 1,3-cyclohexadione (0.0376 g, 0.335 mmol) in acetonitrile (5.0 ml) was added Et₃N (0.070 ml, 0.47 mmol). After stirring for 30 min the reaction mixture was quenched by pouring into 1M HCl (10 ml). The resulting mixture was extracted with CH₂Cl₂ (2×15 ml), the combined organic layers were washed with aq saturated NaHCO₃, dried over anhydrous Na₂SO₄ and evaporated under reduced pressure to afford 3-(3,4-dimethoxyphenyl)-1-methyl-7,8,9,10b-tetrahydro-4aH-chromeno [3,4-d]pyridazine-4,5,10-trione (0.419 g) as a yellow powder. Quantitative NMR analysis using trimethoxybenzene as an internal standard indicates purity of 92% (73% yield).

Alternatively 3-(3 ,4-dimethoxyphenyl)-1-methyl-7,8,9,10b-tetrahydro-4aH-chromeno [3,4-d]pyridazine-4,5,10-trione could be prepared by the following procedure: (3 -oxocyclohexen-1-yl) 2-(3,4-dimethoxyphenyl)-6-methyl-3-oxo-pyridazine-4-carboxylate (0.222 g, 61% purity, 0.35 mmol) and DMAP (0.013 g, 0.11 mmol) were dissolved in MeCN (1.5 mL). After stirring for 30 minutes the reaction mixture was quenched by addition of 1M HCl (1.5 mL). The reaction mixture was extracted with CH₂Cl₂ (3×3.0 mL), the combined organic layers were dried over anhydrous Na₂SO₄ and concentrated under reduced pressure to afford 3-(3,4-dimethoxyphenyl)-1-methyl-7,8,9,10b-tetrahydro-4aH-chromeno [3,4-d]pyridazine-4,5,10-trione (0.270 g) as an amber oil. Quantitative NMR analysis using trimethoxybenzene as an internal standard indicates purity of 43.2% (86% yield).

Alternatively 3-(3,4-dimethoxyphenyl)-1-methyl-7,8,9,10b-tetrahydro-4aH-chromeno [3,4-d]pyridazine-4,5,10-trione could be prepared by the following procedure:

To a solution of 1,3-cyclohexadione (1.825 g, 16.28 mmol) in acetonitrile (8.0 ml) was added triethylamine (2.40 ml, 17.2 mmol) and the reaction mixture was cooled to −18° C. A solution of 2-(3,4-dimethoxyphenyl)-6-methyl-3-oxo-pyridazine-4-carbonyl chloride (5.01 g, 16.23 mmol) in acetonitrile (5.0 ml) was added dropwise while keeping the internal temperature below −15° C. After stirring for 90 min a solution of 1,3-cyclohexadione (0.543 g, 4.85 mmol) and triethylamine (1.10 ml, 8.10 mmol) in acetonitrile (2.0 ml) was added and the reaction mixture was warmed up to 0° C. After stirring at this temperature for 4 h the reaction was quenched by addition of 1M HCl (40 ml). The resulting mixture was extracted with DCM (4×50 ml). The combined organic layers were washed with water (50 ml) and brine (50 ml). Drying over anhydrous Na₂SO₄ and evaporation under reduced pressure afforded 3-(3,4-dimethoxyphenyl)-1-methyl-7,8,9,10b-tetrahydro-4aH-chromeno[3,4-d]pyridazine-4,5,10-trione (6.680 g) as a beige powder. Quantitative NMR analysis using trimethoxybenzene as an internal standard indicates purity of 85% (91% yield).

¹H NMR (400 MHz, CDCl₃): δ 7.03-6.97 (m, 2H), 6.90 (d, J=8.4 Hz, 1H), 4.28 (d, J=7.7 Hz, 1H), 3.90 (s, 3H), 3.90 (s, 3H), 3.70 (d, J=7.7 Hz, 1H), 2.70 (t, J=6.2 Hz, 2H), 2.62-2.55 (m, 2H), 2.27-2.09 (m, 2H), 1.99 (s, 3H).

### Example 9

### 2-[2-(3,4-Dimethoxyphenyl)-6-methyl-3-oxo-pyridazine-4-carbonyl] cyclohexane-1,3-dione

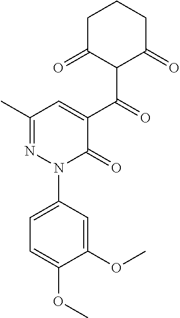 [SMILES: COc1ccc(-n2nc(C)cc(C(=O)C3C(=O)CCCC3=O)c2=O)cc1OC]

To a solution of 3-(3,4-dimethoxyphenyl)-1-methyl-7,8,9,10b-tetrahydro-4aH-chromeno[3,4-d]pyridazine-4,5,10-trione (0.250 g, 85% purity, 0.550 mmol) in 1,2-dichloroethane (2.0 ml) was added 1,3-cyclohexadione (0.0187 g, 0.167 mmol) followed by triethylamine (0.11 ml, 0.80 mmol). The reaction was heated at 60° C. for 4 h, then cooled to ambient temperature and quenched by addition of 1M HCl (2 ml). The resulting mixture was diluted with 1,2-dichloroethane and water. Phases were separated and the aqueous phase was extracted with dichloromethane (3×). The combined organic layers were dried over anhydrous Na₂SO₄ and evaporated under reduced pressure to afford 2-[2-(3,4-dimethoxyphenyl)-6-methyl-3-oxo-pyridazine-4-carbonyl]cyclohexane-1,3-dione (0.268 g) as a yellow oil. Quantitative NMR analysis using trimethoxybenzene as an internal standard indicates purity of 57% (72% yield).

¹H NMR (400 MHz, CDCl₃): δ 16.15 (s, 1H), 7.15-7.11 (m, 1H), 7.10-7.08 (m, 2H), 6.91 (d, J=8.4 Hz, 1H), 3.90 (s, 3H), 3.89 (s, 3H), 2.72 (t, J=6.2 Hz, 2H), 2.48-2.43 (m, 2H), 2.41 (s, 3H), 2.04 (quin, J=6.4 Hz, 2H).

What is claimed is:

1. A process for producing a compound of Formula (I): 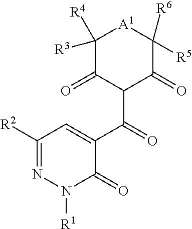
wherein
R¹ is selected from the group consisting of hydrogen, C₁-C₆alkyl, C₁-C₆haloalkyl, C₁-C₃alkoxyC₁-C₃alkyl-, C₁-C₃alkoxyC₂-C₃alkoxyC₁-C₃alkyl-, aryl and a 5 or 6-membered heteroaryl, wherein the heteroaryl contains one to three heteroatoms each independently selected from the group consisting of oxygen, nitrogen and sulphur, and wherein the aryl and heteroaryl component may be optionally substituted;
R² is C₁-C₆ alkyl or C₃-C₆ cycloalkyl;
A¹ is selected from the group consisting of O, C(O) and (CR⁷R⁸);
R⁴, R⁶, R⁷ and R⁸ are each independently selected from the group consisting of hydrogen and C₁-C₄alkyl; and
R³ and R⁵ are each independently selected from the group consisting of hydrogen and C₁-C₄alkyl or together may form a C₁-C₃alkylene chain;
the process comprising
reacting a compound of Formula (XII) 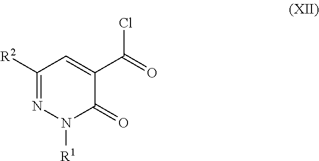
wherein R¹ and R² are as defined with regard to Formula (I);
with a compound of Formula (XIII) 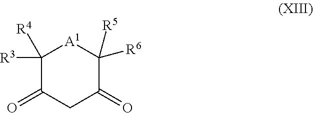
wherein A¹ and R³, R⁴, R⁵ and R⁶ are as defined with regard to Formula (I);
to give a compound of Formula (XIV) 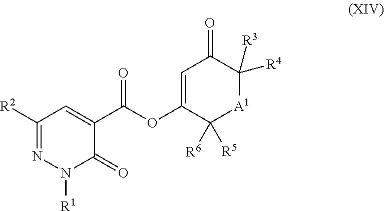
(ii) converting the compound of Formula (XIV) to a compound of Formula (XV) 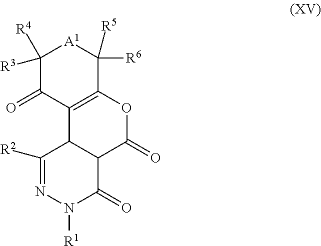
and
(iii) converting the compound of Formula (XV) to a compound of Formula (I) wherein the process is carried out in the presence of a base and in the absence of cyanide ions.

2. A process according to claim 1, wherein R¹ is an optionally substituted heteroaryl.

3. A process according to claim 1, wherein R¹ is an optionally substituted phenyl.

4. A process according to claim 1, wherein R¹ is phenyl optionally substituted by one or two substituents independently selected from the group consisting of halo, C₁-C₄alkyl, C₁-C₄haloalkyl, C₁-C₃ alkoxy, cyano and nitro.

5. A process according to claim 1, wherein A¹ is CR⁷R⁸ and R³, R⁴, R⁵, R⁶, R⁷ and R⁸ are hydrogen.

6. A process according to claim 1, wherein steps (i), (ii) and (iii) are performed in a single operation.

7. A process according to claim 1, wherein the compound of Formula (XII) wherein R¹ is aryl or a 5 or 6-membered heteroaryl as defined in claim 1 is produced by:
(i) reacting together a compound of Formula (V) 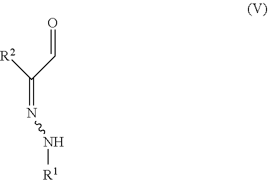
wherein R¹ is aryl or a 5 or 6-membered heteroaryl and R² is as defined in claim 1 with a compound of Formula (XVI) 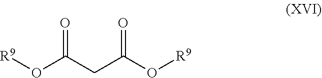
wherein each R⁹ is independently a C₁-C₆alkyl, to give compound of Formula (VI) 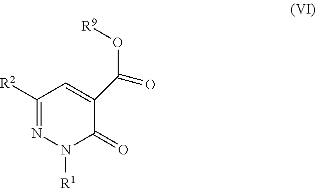
(ii) hydrolysing the compound of Formula VI to a compound of Formula (IX) 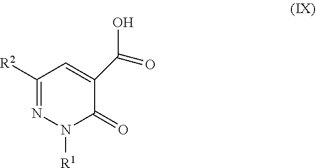
and
(iii) converting the compound of Formula (IX) to the corresponding acid chloride of Formula (XII)

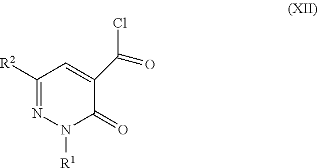

8. A process according to claim 1, wherein the compound of Formula (XII) wherein R¹ is aryl or a 5 or 6-membered heteroaryl and R² is as defined in claim 1 is produced by:
(i) reacting together a Compound of Formula (III) 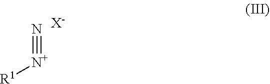
wherein R¹ is aryl or a 5 or 6-membered heteroaryl as defined in claim 1 and X is selected from the group consisting of Cl, Br and HSO₄ with a compound of Formula (X) 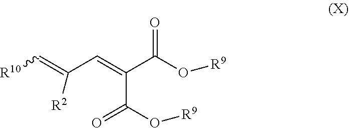
wherein R² is as defined in claim 1, R¹⁰ is NMe₂, NEt₂, OH or C₁-C₃alkoxy and each R⁹ is independently a C₁-C₆alkyl, to give a compound of Formula (XI) 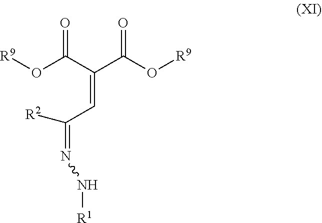
(ii) cyclising the compound of Formula (XI) to a compound of Formula (VI) 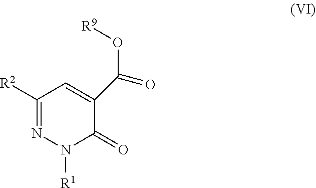
(iii) hydrolysing the compound of Formula (VI) to a compound of Formula (IX) 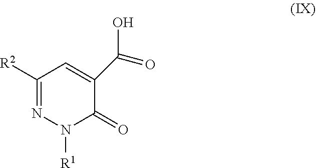
and
(iv) converting the compound of Formula (IX) to the corresponding acid chloride of Formula (XII)

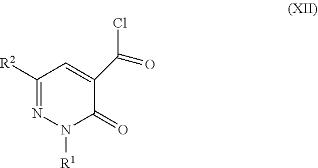

9. A process according to claim 1, wherein R¹ is 3,4-dimethoxyphenyl.

10. A process according to claim 1, wherein R² is methyl.

11. A compound of Formula (XV) 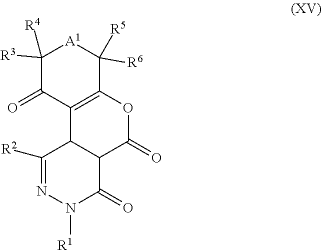
wherein R¹, R², R³, R⁴, R⁵, R⁶ and A¹ are as defined in claim 1.

12. A compound of Formula (XVa)

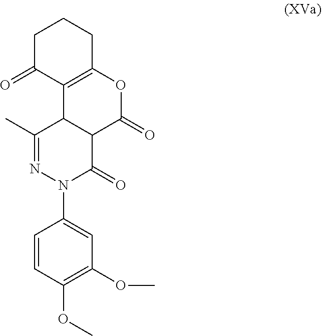

13. A compound of Formula (Va)

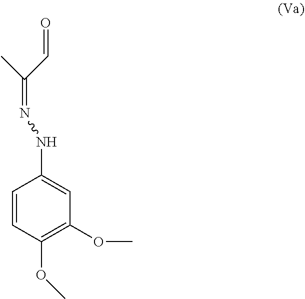 [SMILES: COc1ccc(NN=C(C)C=O)cc1OC]

14. A compound of Formula (XIa) 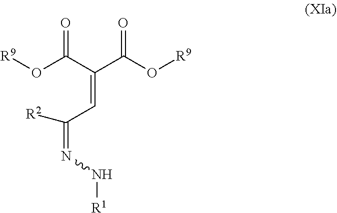
wherein R⁹ are both methyl or ethyl, R² is methyl and R¹ is 3,4-dimethoxyphenyl-.
